(19) United States
(12) Patent Application Publication
(10) Pub. No.: US 2015/0010953 A1
(43) Pub. Date: Jan. 8, 2015

(54) METHOD FOR PRODUCING A POPULATION OF OLIGONUCLEOTIDES THAT HAS REDUCED SYNTHESIS ERRORS

(71) Applicant: AGILENT TECHNOLOGIES, INC., Loveland, CO (US)

(72) Inventors: Derek Lee Lindstrom, San Carlos, CA (US); Jeffrey R. Sampson, San Francisco, CA (US); Daniel E. Ryan, San Francisco, CA (US)

(21) Appl. No.: 13/935,201

(22) Filed: Jul. 3, 2013

(51) Int. Cl. C12N 15/10; C12P 19/34

(52) U.S. Cl. CPC C12N 15/1006; C12P 19/34
USPC 435/91.52; 435/91.5; 435/196; 435/270; 536/25.41

(57) ABSTRACT

Provided herein is a method for producing a population of oligonucleotides that has reduced synthesis errors. In certain embodiments, the method comprises: a) obtaining an initial population of hairpin oligonucleotide molecules that each comprise a double-stranded stem region and a loop region; b) contacting the double-stranded region of the hairpin oligonucleotide molecules with a mismatch binding protein; and c) eliminating any molecules that bind to the mismatch binding protein, thereby producing a population of oligonucleotides that has reduced synthesis errors. A kit and a composition for performing the method are also provided.

### BACKGROUND

Many oligonucleotide synthesis methods are imperfect in that they result in a population of oligonucleotides that have a variety of synthesis errors, e.g., nucleotide substitutions and deletions. The method described below provides a way in which oligonucleotides that have synthesis errors can be eliminated enzymatically, thereby producing a population of oligonucleotides that has reduced synthesis errors.

### SUMMARY

Provided herein is a method for producing a population of oligonucleotides that has reduced synthesis errors. In certain embodiments, the method comprises: a) obtaining an initial population of hairpin oligonucleotide molecules that each comprise a double-stranded stem region and a loop region; b) contacting the double-stranded region of the hairpin oligonucleotide molecules with a mismatch binding protein; and c) eliminating any molecules that bind to the mismatch binding protein, thereby producing a population of oligonucleotides that has reduced synthesis errors.

A kit for performing the method is also provided. In certain embodiments, the kit comprises: a) a population of hairpin oligonucleotide molecules that each comprise a double-stranded stem region and a loop region; and b) a mismatch binding protein.

Also provided is a composition. In certain embodiments, the composition comprises: a) a population of hairpin oligonucleotide molecules that each comprise a double-stranded stem region and a loop region; and b) a mismatch binding protein, where the mismatch binding protein binds to hairpin oligonucleotide molecules that have a synthesis error in the double-stranded stem region.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates some of the general principles of one embodiment of the subject method.

FIG. 2 schematically illustrates some of the general principles of another embodiment of the subject method.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) and/or deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length, for example.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides. The primers herein are selected to be substantially complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementary with the sequence of the strand to hybridize therewith and thereby form the template for the synthesis of the extension product.

The term “hybridization” or “hybridizes” refers to a process in which a nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “Selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions include hybridization at about 42 C in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “duplex,” or “duplexed,” as used herein, describes two complementary polynucleotides that are base-paired, i.e., hybridized together.

The term “amplifying” as used herein refers to the process of synthesizing nucleic acid molecules that are complementary to one or both strands of a template nucleic acid. Amplifying a nucleic acid molecule typically includes denaturing the template nucleic acid, annealing primers to the template nucleic acid at a temperature that is below the melting temperatures of the primers, and enzymatically elongating from the primers to generate an amplification product. The denaturing, annealing and elongating steps each can be performed once. Generally, however, the denaturing, annealing and elongating steps are performed multiple times (e.g., at least 5 or 10 times, up to 30 or 40 or more times) such that the amount of amplification product is increasing, often times exponentially, although exponential amplification is not required by the present methods. Amplification typically requires the presence of deoxyribonucleoside triphosphates, a DNA polymerase enzyme and an appropriate buffer and/or co-factors for optimal activity of the polymerase enzyme. The term “amplification product” refers to the nucleic acid sequences, which are produced from the amplifying process as defined herein.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

The term “free in solution,” as used here, describes a molecule, such as a polynucleotide, that is not bound or tethered to another molecule.

The term “ligating”, as used herein, refers to the enzymatically catalyzed joining of the terminal nucleotide at the 5′ end of a first DNA molecule to the terminal nucleotide at the 3′ end of a second DNA molecule.

A “plurality” contains at least 2 members. In certain cases, a plurality may have at least 10, at least 100, at least 100, at least 10,000, at least 100,000, at least 10⁶, at least 10⁷, at least 10⁸ or at least 10⁹ or more members.

If two nucleic acids are “complementary”, they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

The term “digesting” is intended to indicate a process by which a nucleic acid is cleaved by a restriction enzyme. In order to digest a nucleic acid, a restriction enzyme and a nucleic acid containing a recognition site for the restriction enzyme are contacted under conditions suitable for the restriction enzyme to work. Conditions suitable for activity of commercially available restriction enzymes are known, and supplied with those enzymes upon purchase.

A “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds. A hairpin molecule contains two complementary strands that are separated by a loop region.

In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure.

The term “denaturing,” as used herein, refers to the separation of at least a portion of the base pairs of a nucleic acid duplex by placing the duplex in suitable denaturing conditions. Denaturing conditions are well known in the art. In one embodiment, in order to denature a nucleic acid duplex, the duplex may be exposed to a temperature that is above the Tm of the duplex, thereby releasing one strand of the duplex from the other. In certain embodiments, a nucleic acid may be denatured by exposing it to a temperature of at least 90° C. for a suitable amount of time (e.g., at least 30 seconds, up to 30 mins). In certain embodiments, fully denaturing conditions may be used to completely separate the base pairs of the duplex. In other embodiments, partially denaturing conditions (e.g., with a lower temperature than fully denaturing conditions) may be used to separate the base pairs of certain parts of the duplex (e.g., regions enriched for A-T base pairs may separate while regions enriched for G-C base pairs may remain paired.) Nucleic acid may also be denatured chemically (e.g., using urea or NaOH).

The term “extending”, as used herein, refers to the extension of a primer by the addition of nucleotides using a polymerase. If a primer that is annealed to a nucleic acid is extended, the nucleic acid acts as a template for extension reaction.

The term “population of oligonucleotides”, as used herein, refers to a composition of matter that contains a plurality of oligonucleotide molecules. A population may be composed of oligonucleotide molecules of substantially the same sequence (i.e., with the exception of oligonucleotide molecules that contain synthesis errors), or a mixture of oligonucleotides of different sequences. A mixture of oligonucleotides of different sequences may be made by synthesizing different oligonucleotides separately (e.g., on one or more solid supports) and then mixing them together.

The term “synthesis error”, as used herein, refers to an error in the synthesis of an oligonucleotide. A synthesis error can be in the form of a mis-incorporation (which results in an oligonucleotide that has one or more nucleotide substitutions relative to the nucleotide sequence of the desired product), a failure to incorporate (which results in an oligonucleotide that is shorter than the desired product) or an extra incorporation (which results in an oligonucleotide that is longer than the desired product).

The term “mismatch”, as used herein, refers to any type of imperfect or unmatched base-pairing in a double stranded nucleic acid, including those generated by nucleotide substitutions, insertions or deletions in one strand of a double stranded nucleic acid relative to the complement of the other. A mismatch, a region of imperfect complementarity, occurs between two regions of complementarity in a double stranded nucleic acid. An insertion or deletion of a nucleotide in one of the strands may cause a “bulge” in a double stranded nucleic acid. In certain cases, a double stranded nucleic acid that contains a mismatch may be referred to as a “heteroduplex”, i.e., an imperfect duplex.

The term “mismatch binding protein”, as used herein, refers to any protein (including peptides) that binds to and optionally cleaves a mismatch in a double stranded nucleic acid. In certain cases, a mismatch binding protein may be derived from a protein that is involved in DNA repair in a cell. Bacterial MutS protein, E. coli endonuclease V, a eukaryotic MSH protein, T4 endonuclease VII, T7 endonuclease I, bacterial mutH, or celery CelI and are non-limiting examples of such proteins.

The term “variant”, as used herein, refers to a modified protein has an amino acid sequence that is at least 80% identical (e.g., at least 90%, at least 95%, at least 98% or at least 99%) identical to the amino acid sequence of a wild type protein that has at least some of the same activity as the wild type protein. In certain cases, a variant may have changes in its amino acid sequence that result in a decrease in an undesirable activity. In certain cases, a variant may be a variant of a bacterial MutS protein, E. coli endonuclease V, a eukaryotic MSH protein, T4 endonuclease VII, T7 endonuclease I, bacterial mutH, or celery CelI, or a functional ortholog thereof.

The term “hairpin oligonucleotide molecules”, as used herein, refers to oligonucleotide molecules that have a self-complementary region such that the oligonucleotides fold to form a hairpin structure. As is well known, a hairpin contains a double stranded stem region and a loop region that is single stranded. The strands of the double stranded stem may be perfectly complementary or may contain one or more mis-matches.

The term “eliminating”, as used herein, refers to any way for preventing an oligonucleotide from participating in a future reaction. The term “eliminating” is intended to encompass cleaving an oligonucleotide as well as physically removing an oligonucleotide molecule from a population.

The term “cleaving”, as used herein, refers to the cleavage of a phosphodiester bond in the backbone of a nucleic acid.

The term “the site of a mismatch”, as used herein, in the context of cleaving at the site of a mismatch, refers to a cleavage of a phosphodiester bond that occurs near to a mismatch, e.g., at a bond that is connected to a mis-matched nucleotide, or a bond that is connected to a nucleotide that is near a mis-matched nucleotide (e.g., one two or three nucleotides upstream or downstream from a mis-matched nucleotide). Many enzymes cleave both strands at the site of a mismatch.

The term “synthon”, as used herein, refers to a synthetic nucleic acid that has been assembled in vitro from several shorter nucleic acids, e.g., oligonucleotides. A synthon can be made by polymerase chain assembly (PCA), as used herein or ligase chain assembly (LCA), for example.

The term “polymerase chain assembly”, as used herein, refers to a protocol in which multiple overlapping oligonucleotides are combined and subjected to multiple rounds of primer extension (i.e., multiple successive cycles of primer extension, denaturation and renaturation in the presence of a polymerase and nucleotides) to extend the oligonucleotides using each other as a template, thereby producing a product molecule. In many cases, the final product molecule is amplified using primers that bind to sites at the ends of the product molecule, and the product molecule is digested with one or more restriction enzymes and cloned. Polymerase chain assembly may include additional steps, such as digestion of the product molecule with a restriction enzyme to, e.g., prepare the product molecule for cloning.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

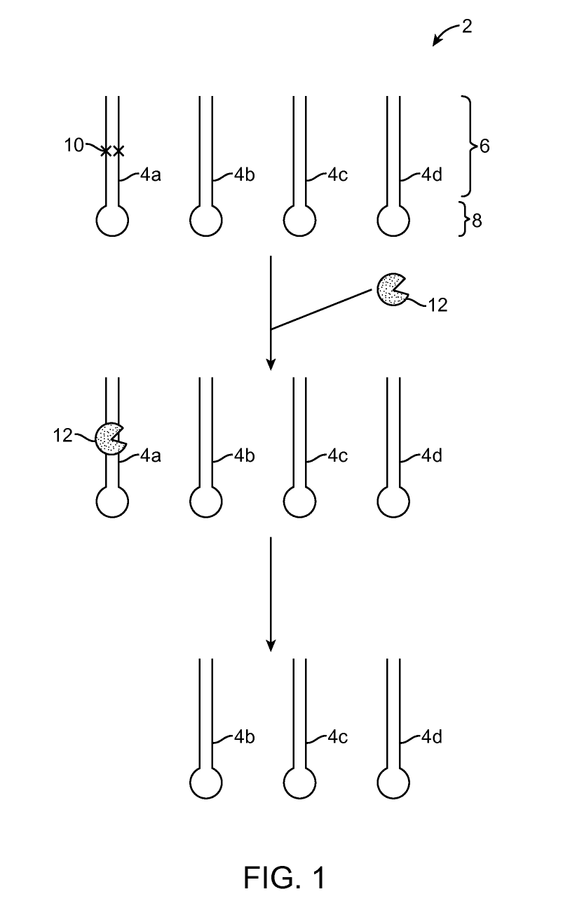

With reference to FIG. 1, one embodiment of the method involves obtaining: a) obtaining an initial population of hairpin oligonucleotide molecules 2. As shown, the initial population of hairpin oligonucleotide molecules 2 is composed of 4 molecules: hairpin oligonucleotide molecules 4a, hairpin oligonucleotide molecules 4b, hairpin oligonucleotide molecules 4c and hairpin oligonucleotide molecules 4d. In practice, the population can contain at least 10⁶, at least 10⁸, at least 10¹⁰ or at least 10¹² oligonucleotide molecules. In some embodiments, the nucleotide sequences of the oligonucleotides molecules can be the same as one another. In other embodiments, population can contain oligonucleotides molecules that have different sequences. As shown, each of the hairpin oligonucleotide molecules comprises a double-stranded stem region 6 and a loop region 8. In the example shown, oligonucleotide molecule 4a has a mismatch 10 in its double stranded stem region. The mismatch is caused by an error in the synthesis of that oligonucleotide. In practice, every population of oligonucleotides contains synthesis errors, and the number of molecules that have errors depends on how the long the oligonucleotides are and how they were made, among other things. In some cases, particularly with longer oligonucleotides (i.e., oligonucleotides that are over 50 bases in length), at least 50% (e.g., at least 70%, at least 80%, at least 90% or at least 95%) of the oligonucleotide molecules in a population may contain at least one synthesis error.

After the initial population of hairpin molecules has been obtained, the double stranded region of those molecules is contacted with a mismatch binding protein 12, i.e., a protein that specifically binds to double stranded regions that contain a mismatch, but not to perfectly complementary double stranded regions. As shown, only oligonucleotide molecule 4a has a mismatch and, as such, the mismatch binding protein recognizes only that oligonucleotide.

Next, the method comprises eliminating any molecules that bind to the mismatch binding protein. This step of the method results in a population of oligonucleotides that has reduced synthesis errors 14. This step of the method may be implemented in a variety of different ways. In one embodiment, the eliminating is done by separating any molecules that bind to the mismatch binding protein from the remainder of the molecules by immobilizing the mismatch binding protein on a solid support. This step may be done using an antibody that binds to the mismatch binding protein, a biotinylated mismatch binding protein, or a mismatch binding protein that is attached magnetic beads, for example. In one embodiment, the double stranded region of the oligonucleotides is contacted to a mismatch binding protein in solution under conditions by which the protein binds to any oligonucleotides that contain a mismatch, and then removing any oligonucleotides that are bound to the mismatch binding protein from the solution by contacting the solution with a solid support that contains a reagent that has affinity for the protein (e.g., streptavidin or an antibody, etc.) or some other means, e.g., magnetism (if the protein is linked to a magnetic bead, for example). In these embodiments, the eliminating is done by separating any molecules that bind to the mismatch binding protein from the remainder of the molecules by immobilizing the mismatch binding protein on a solid support.

In other embodiments, the mismatch binding protein is an endonuclease and the eliminating is done by cleaving the double-stranded region at the site of a mismatch. In these embodiments, the endonuclease may cut both strands of the double stranded region at the site of the mismatch, thereby eliminating full length oligonucleotides from future steps.

In the example shown in FIG. 1, the population of hairpin oligonucleotide molecules still have their loop regions when they are contacted with the mismatch binding protein. However, in practice, the loop regions of the oligonucleotides may be removed (e.g., by cleavage of a site within the oligonucleotides or using a restriction enzyme that recognizes a site in the double stranded region of the oligonucleotides, where the site is proximal to the loop) prior to or after contacting the oligonucleotides with the mismatch binding protein. In certain embodiments and as described in greater detail below, the mismatch binding protein may by an endonuclease and may recognize the loop region in addition to the site of the mismatch. In these embodiments, the mismatch binding protein may cleave the loop region in addition to cleaving the double stranded region of any molecules that contain a mismatch. In certain embodiments, the method comprises cleaving the loop region from the hairpin oligonucleotide molecules prior to the contacting step.

Mismatch recognition can be accomplished by the action of any suitable protein (such as bacterial MutS proteins, eukaryotic MSH proteins, T4 endonuclease VII, T7 endonuclease I, and celery CelI, or a variant thereof, for example). In some embodiments, a mismatch binding protein such as MutS can be used to bind to a mismatch in the double stranded region, thereby providing a way by which oligonucleotides that contain a mismatch can be removed from solution. MutS is a bacterial protein. MutS from Thermus aquaticus can be purchased commercially from the Epicenter Corporation, Madison, Wis., Catalog No. SP72100 and SP72250. The gene sequence for the protein is also known and published in Biswas and Hsieh, Jour. Biol. Chem. 271:5040-5048 (1996) and is available in GenBank, accession number U33117. In other embodiments, T7 endonuclease I specifically cleaves a DNA strand at a mismatch, and it would be possible to use this enzyme as a catalytic destroyer of mismatched sequences or to inactivate the cleavage function of this enzyme for use in this process as a mismatch binding agent. Likewise, T4 endonuclease VII can specifically bind and cleave DNA at duplex mismatches (Ya et al Genomics 1995 32: 431-435). A mutant version of this enzyme has already been engineered that lacks the nuclease activity but retains the ability to bind mutant duplex DNA molecules (see Golz and Kemper, Nucleic Acids Research, 27:e7 (1999)). In other embodiments, a mismatch specific endonuclease, such as CEL1 can be used to cleave mismatch containing hybrids (see for example, PCT Patent Application No. PCT/US2010/057405, which is incorporate herein by reference in its entirety). Heteroduplex recognition and cleavage can be achieved by applying a mismatch endonuclease to the reaction mix. CEL1 endonuclease has high specificity for insertions, deletions and base substitution mismatches and can detect two polymorphisms which are five nucleotides apart form each other. CEL1 is a plant-specific extracellular glycoprotein that can cleave heteroduplex DNA at all possible single nucleotide mismatches, at 3′ to the mismatches (Oleykowski C A et al, 1998, Nucleic Acids Res. 26: 4596-4602; Yang (Biochemistry 1999 39: 3533-3541). CEL1 is useful in mismatch detection assays that rely on nicking and cleaving duplex DNA at insertion/deletion and base substitution mismatches. In an exemplary embodiment, a SURVEYER™ nuclease (Transgenomic Inc.) could be used. This nuclease is a mismatch specific endonuclease that cleaves all types of mismatches such as single nucleotide polymorphisms, small insertions or deletions. Further suitable enzymes may be described in Biswas (J. Biochem. 1997 272 13355-13364); Eisen (Nuc. Acids Res. 1998 26: 4291-4300); Beaulieu (Nuc. Acids Res. 2001 29: 1114-1124); Smith (Proc. Natl. Acad. Sci. 1997 94: 6847-6850); Smith (Proc. Natl. Acad. Sci. 1996 93: 4374-4379); Bjornson (J. Biochem 2003 278: 18557-18562), U.S. Pat. No. 6,008,031, U.S. Pat. No. 5,922,539, US5,861, US482, U.S. Pat. No. 5,858,754, U.S. Pat. No. 5,702,894, U.S. Pat. No. 5,679,522, U.S. Pat. No. 5,556,750, and U.S. Pat. No. 5,459,039, all of which are hereby incorporated by reference.

The various parts of a subject hairpin oligonucleotide may be of any suitable length depending on the desired application. For example, the loop region may be a single nucleotide in length, two nucleotides, three nucleotides, or 4-20 nucleotides or more in length. The double stranded region may be 20 to 200 or more nucleotides in length, e.g., 30-100 nucleotides in length. In one embodiment, the hairpin of a subject oligonucleotide may have a Tₘ of over 70° C. (e.g., a Tₘ of at least 80° C., at least 90° C., at least 95° C. or at least 100° C.) and in certain cases may contain an “unmeltable hairpin” such as that described in e.g., Varani et al (Exceptionally stable nucleic acid hairpins Annu Rev Biophys Biomol Struct. 1995.;24:379-404). In a particular cases, a hairpin adaptor may contain the sequence d(GCGAAGC), which forms very a stable hairpin with a melting temperatures of above 70° C. (Padtra et al Refinement of d(GCGAAGC) hairpin structure using one- and two-bond residual dipolar couplings J. Biomol. NMR. 2002 24:1-14). As noted above, the double stranded region may contain other useful sequences (such as a restriction site (which would allow the hairpin to be cleaved from the double stranded region), a sequencing primer binding site and/or a PCR primer binding site, etc.) that can be used in later steps in the method.

In one embodiment, the loop itself may contain a modified nucleotide or nucleotide linkage that allows the loop to be specifically cleaved. For example, the loop may contain a uracil reside so that can be cleaved by uracil-DNA Glycosylase (UDG), which efficiently catalyses the release of free uracil from uracil-containing DNA, or the loop can contain one or more ribonucleotides that can be cleaved by an RNA-specific nuclease, e.g., RNase A or RNase I, which cleaves single stranded RNA. Alternatively, the loop may contain a cleavable bond, such as, but are not limited to, the following: base-cleavable sites such as esters, particularly succinates (cleavable by, for example, ammonia or trimethylamine), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Other cleavable bonds will be apparent to those skilled in the art or are described in the pertinent literature and texts (e.g., Brown (1997) Contemporary Organic Synthesis 4(3); 216-237). In particular embodiments, a photocleavable linker (e.g., a uv-cleavable linker) may be employed. Suitable photocleavable linkers for use in may include ortho-nitrobenzyl-based linkers, phenacyl linkers, alkoxybenzoin linkers, chromium arene complex linkers, NpSSMpact linkers and pivaloylglycol linkers, as described in Guillier et al (Chem. Rev. 2000 Jun. 14; 100(6):2091-158).

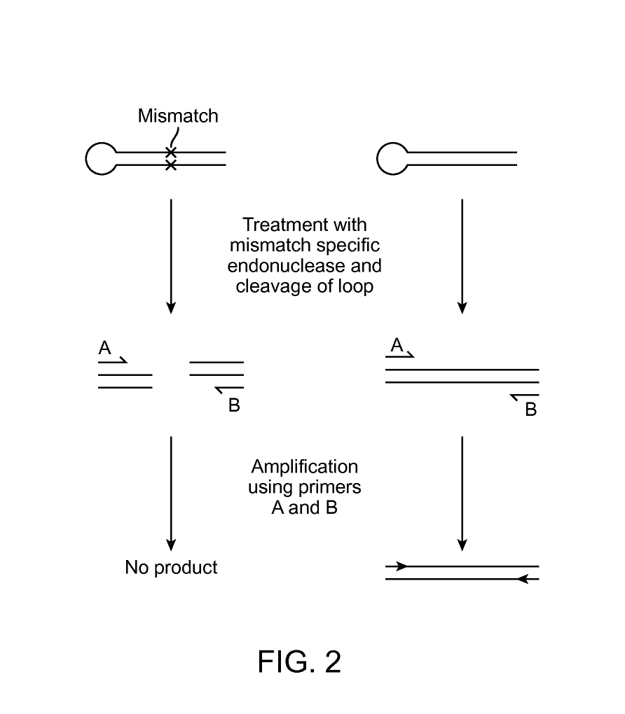

In some embodiments, the method may comprising amplifying a sequence in the double stranded stem region, after the loop region has been cleaved, using oligonucleotide primers that bind to the ends of the double stranded region of the hairpin oligonucleotides. In embodiments that rely on a mismatch binding protein that has endonuclease activity to cleave both strands of the double stranded region at a mismatch, oligonucleotides that have mismatches are cleaved and therefore cannot be amplified. The principle of this part of the method is illustrated in FIG. 2. As would be apparent, the amplification product produced by this embodiment of the method may contain primer sites at their ends. If desired, the primer sites can be cleaved from the product using a restriction enzyme. In particular embodiments, a Type IIs restriction enzyme (i.e., a restriction enzyme that cuts upstream or downstream from its recognition site) may be used to remove the primer sites. In these embodiments, the double stranded region of the hairpin oligonucleotides may be designed to contain recognition sites for one or more Type IIs restriction enzyme so that any primer sites and the recognition sites the Type IIs restriction enzymes can be cleaved from the amplification product prior to use in the next step of the method.

In certain embodiments, the initial population of hairpin oligonucleotide molecules comprises multiple different hairpin oligonucleotides molecules that have the same loop region and different double-stranded stem regions. The initial population of hairpin oligonucleotides may contain any number, e.g., one to one million, different species of oligonucleotides (i.e., oligonucleotides having a different sequence). In certain cases, the initial population of hairpin oligonucleotide molecules may contain at least 10, at least 100, at least 1,000 or at least 10,000 or more different species of oligonucleotide (i.e., oligonucleotides having a different sequence). In certain cases, a population of oligonucleotides can be made by fabricating an array of the oligonucleotides using in situ synthesis methods, and cleaving oligonucleotides from the substrate. Examples of such methods are described in, e.g., Cleary et al (Nature Methods 2004 1: 241-248) and LeProust et al (Nucleic Acids Research 2010 38: 2522-2540). In some embodiments, the sequences of some of the double-stranded stem regions of the different hairpin oligonucleotides may be at least 80% identical to one another, e.g., at least 90%, at least 95%, at least 98%, at least 99%, identical to one another) and, as such, would otherwise cross-hybridize to one another if they were annealed in solution. Provision of a hairpin in each of the oligonucleotides allows the selective elimination of oligonucleotides that contain a mismatch, without any need to hybridize to other oligonucleotides that have the “correct” sequence.

In some embodiments, the oligonucleotides in the population are designed such that after cleavage of the loops and/or amplification of the double stranded region, the products can be assembled into a synthon. The method for eliminating error-containing oligonucleotides from a population, as described above, finds particular use in such a method because the intrinsic error rate of each coupling step in oligonucleotide synthesis (which is typically below 0.5%) is such that preparations of longer oligonucleotides are increasingly likely to be riddled with errors, and that a synthon made from such oligonucleotides will be numerically overwhelmed by sequences containing errors. Errors in gene synthesis are typically controlled in two ways: 1) the individual oligonucleotides can each be purified to remove error sequences; 2) the final cloned products are sequenced to discover if errors are present. In this latter case, the errors are dealt with by either sequencing many clones until an error-free sequence is found, using mutagenesis to specifically fix an error, or choosing and combining specific error-free sub-sequences to build an error free full length sequence. The method described above decreases the need for oligonucleotide purification and the need to screen candidate synthons to identify one with the correct sequence.

Assembly of a synthon may be done using polymerase chain assembly (PCA), i.e., a protocol in which multiple overlapping oligonucleotides are combined and subjected to multiple rounds of primer extension (i.e., multiple successive cycles of primer extension, denaturation and renaturation in the presence of a polymerase and nucleotides) to extend the oligonucleotides using each other as a template, thereby producing a product molecule. Suitable conditions for performing polymerase chain assembly are found in, e.g., Hughes, et al. (Methods in Enzymology 2011 498:277-309) and Wu, et al. (J. Biotechnol. 2006 124:496-503). This step may also be done by ligase chain assembly (LCA), which essentially involves annealing multiple oligonucleotides to one another, ligating the ends of the annealed oligonucleotides to one another, and then amplifying the resultant product. Other non-PCA-based methods for assembling synthons from oligonucleotides are described in Xiong et al (Biotechnol. Adv. 2008 26: 121-134), which is incorporated by reference herein for disclosure of those methods. Methods for gene assembly are also described in, e.g., Au et al (Biochem. Biophys. Res. Comm. 1998 248, 200-203); Baedeker (FEBS Letters 1999 475: 57-60), Casimiro (Structure 1997 5: 1407-1412); Cello (Science 2002 297: 1016-1018); Kneidinger (Biotechniques 2001 30: 249-252); Dietrich (Biotech. Techniques 1998 12: 49-54); Hoover (Nuc. Acids Res 2002 30:1-7); Stemmer (Gene 1995 164: 49-53); Withers-Martinez (Protein Eng. 1999 12: 1113-1120); U.S. Pat. No. 6,521,453, U.S. Pat. No. 6,521,427, US20030165946, US20030138782, and US20030087238 and all of which are hereby incorporated by reference.

The method described above finds particular use in the multiplexed assembly of a reduced-error population of oligonucleotides into a plurality of different high fidelity synthons, wherein the synthons have nucleotide sequences that are at least at least 80% identical to one another (e.g., at least 90% identical, at least 95% identical, at least 98% identical or at least 99% identical to one another). In these embodiments, the assembly may be multiplexed such that several different synthons (e.g., 2-100 synthons or more) that are variants of one another are assembled in a single reaction. Certain embodiments may be used to assemble multiple synthons in the same reaction vessel. For example, certain embodiments may be used assemble at least 2, at least 5, at least 10, at least 50, at least 100, at least 500, at least 1,000 or more synthons in the same reaction vessel. The embodiment described may be particularly useful for assembling, in the same reaction vessel, several variants of the same sequence, where the sequences of the variants are similar to one another.

A synthon itself can be of any sequence and, in certain cases, may encode a sequence of amino acids, i.e., may be a coding sequence. In other embodiments, the synthon can be a regulatory sequence such as a promoter or enhancer. In particular cases, the synthon may encode a regulatory RNA. In certain cases a synthon may have a biological or structural function.

In particular cases, synthons may be cloned into a vector that provides for expression of the synthon in a cell. In these embodiments, the expression vector may contain a promoter, terminator and other necessary regulatory elements to effect transcription and in certain cases translation of the synthon, either as a single protein, or as a fusion with another protein. In these embodiments, the method may further comprises transferring the expression vector into a cell to produce the expression product (e.g., a protein) encoded by the synthon. This embodiment of the method may comprise screening the expression product for an activity.

The method described above may be used to prepare high fidelity oligonucleotides for other uses in addition their use in making high fidelity synthons. For example, on might employ high-fidelity pools of oligonucleotides for site-directed mutagenesis, for multiplex genome engineering and accelerated evolution (e.g., MAGE; Wang et al, Nature. 2009 460: 894-8); or to produce sequences encoding siRNAs or shRNAs. High fidelity oligonucleotides may be used in a variety of medical applications.

Also provided is composition produced by the method described above. In certain embodiments, the composition may comprise a) a population of hairpin oligonucleotide molecules that each comprise a double-stranded stem region and a loop region; and b) a mismatch binding protein; wherein the mismatch binding protein is bound to any hairpin oligonucleotide molecules that have a synthesis error in the double-stranded stem region.

In one example, a plurality of different double stranded oligonucleotides is synthesized on the surface of a solid support. Each oligonucleotide is synthesized as a hairpin that contains a double stranded region and a short hairpin loop sequence (for example, the 7 bp hairpin described by Hirao et al, Nucleic Acids Res. 1994 22: 576-82). During synthesis, protecting groups on each base of the growing oligonucleotide preclude formation of double stranded DNA structures. After synthesis is complete, the oligonucleotide is deprotected and cleaved from the solid substrate in a single chemical step. The deprotected oligonucleotide spontaneously forms single-molecule hairpin DNA in solution. If a single molecule contains the correct nucleotide for both plus and minus strands, the double stranded DNA region of the molecule will be a homoduplex. If one or both strands contain a synthesis error, the molecule will be a heteroduplex with one or more mismatched bases. After hairpin formation, the entire library of oligonucleotides will be treated with a mismatch-specific nuclease, e.g., T7 endonuclease I, which cleaves both the plus and minus DNA strands adjacent to mismatched bases. The mismatch-specific nuclease can perform two functions. First, it will recognize and cleave both strands of any heteroduplex DNA, dramatically reducing the number of error-containing double-stranded molecules. Second, the enzyme should recognize the hairpin loop in all molecules and cleave both the plus and minus strands at positions that are adjacent to the loop. This will effectively process each single molecule into a homoduplex double-stranded DNA that is the starting reagent for any method that needs high fidelity, e.g., gene assembly methods. In certain cases, the oligonucleotides can be designed to contain a restriction endonuclease site between the hairpin and desired sequence such that the hairpin molecules can be cleaved by the restriction enzyme before treatment by the mismatch-specific nuclease. This embodiments would provide a predictable end sequence (ie a specific sequence or single-stranded overhang) that may be useful for downstream processing.

### Kits

Also provided by this disclosure is a kit for practicing the subject method, as described above. A subject kit may contain at least: a) a population of hairpin oligonucleotide molecules that each comprise a double-stranded stem region and a loop region; and b) a mismatch binding protein. In particular cases, the sequences of the double-stranded stem regions of the hairpin oligonucleotide molecules are at least 80% identical to one another. In some cases, the mismatch binding protein is T7 endonuclease I, mutS or a variant thereof. In particular cases, the hairpin oligonucleotide molecules comprise a site for a restriction enzyme in the double stranded region, proximal to the loop and the kit further comprises the restriction enzyme. The kit may also comprise reagents (e.g., polymerase, nucleotides, ligase, etc.) for assembling the products of the method described above into one or more synthons. The various components of the kit may be present in separate containers or certain compatible components may be pre-combined into a single container, as desired.

In addition to above-mentioned components, the subject kits may further include instructions for using the components of the kit to practice the subject methods, i.e., to provide instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EMBODIMENTS

A method for producing a population of oligonucleotides that has reduced synthesis errors is provided. In certain embodiments, the method comprises a) obtaining an initial population of hairpin oligonucleotide molecules that each comprise a double-stranded stem region and a loop region; b) contacting the double-stranded region of the hairpin oligonucleotide molecules with a mismatch binding protein; and c) eliminating any molecules that bind to the mismatch binding protein, thereby producing a population of oligonucleotides that has reduced synthesis errors. In any embodiment, the method may comprise cleaving the loop region from the hairpin oligonucleotide molecules prior to step b). In any embodiment, the eliminating may be done by separating any molecules that bind to the mismatch binding protein from the remainder of the molecules by immobilizing the mismatch binding protein on a solid support. In any embodiment, the mismatch binding protein may be bacterial MutS protein, E. coli endonuclease V, a eukaryotic MSH protein, T4 endonuclease VII, T7 endonuclease I, bacterial mutH, or celery CelI, or a variant thereof. In any embodiment, the mismatch binding protein may be an endonuclease and the eliminating is done by cleaving the double-stranded region at the site of a mismatch. In any embodiment, the endonuclease may also cleave the loop region. In any embodiment the method may further comprise, after the loop region has been cleaved, amplifying a sequence in the double stranded stem region using oligonucleotide primers that bind to the ends of the double stranded region of the hairpin oligonucleotides. In any embodiment, the initial population of hairpin oligonucleotide molecules may comprise multiple different hairpin oligonucleotides molecules that have the same loop region and the same or different double-stranded stem regions. In any embodiment, the sequences of the double-stranded stem regions of the multiple different hairpin oligonucleotides may be at least 80% identical to one another. In any embodiment, the method may further comprise assembling the reduced-error population of oligonucleotides into a plurality of different synthons, wherein the synthons are at least at least 80% identical to one another. Assembly may be done by polymerase chain assembly (PCA) or ligase chain assembly (LCA), for example. In any embodiment, the hairpin oligonucleotide molecules comprise a site for a restriction enzyme in the double stranded region, proximal to the loop. In any embodiment, the loop region of the hairpin oligonucleotide molecules is at least four nucleotides in length. In any embodiment, the double-stranded region of the hairpin oligonucleotide molecules is at least 20 nucleotides in length.

Also provided is a kit. In certain embodiments the kit comprises a) a population of hairpin oligonucleotide molecules that each comprise a double-stranded stem region and a loop region; and b) a mismatch binding protein. In any embodiment, the mismatch binding protein may be bacterial MutS protein, E. coli endonuclease V, a eukaryotic MSH protein, T4 endonuclease VII, T7 endonuclease I, bacterial mutH, or celery CelI, or a variant thereof. In any embodiment, the sequences of the double-stranded stem regions of the hairpin oligonucleotide molecules are at least 80% identical to one another. In any embodiment, the hairpin oligonucleotide molecules may comprise a site for a restriction enzyme in the double stranded region, proximal to the loop and the kit may further comprises the restriction enzyme. In any embodiment, the mismatch binding protein may be an endonuclease that cleaves the double-stranded region at the site of a mismatch. In any embodiment, the endonuclease may also cleave the loop region. In any embodiment, the initial population of hairpin oligonucleotide molecules may comprise multiple different hairpin oligonucleotides molecules that have the same loop region and the same or different double-stranded stem regions. In any embodiment, the sequences of the double-stranded stem regions of the multiple different hairpin oligonucleotides may be at least 80% identical to one another. In any embodiment, the loop region of the hairpin oligonucleotide molecules is at least four nucleotides in length. In any embodiment, the double-stranded region of the hairpin oligonucleotide molecules is at least 20 nucleotides in length.

What is claimed is:

1. A method for producing a population of oligonucleotides that has reduced synthesis errors, comprising:
a) obtaining an initial population of hairpin oligonucleotide molecules that each comprise a double-stranded stem region and a loop region;
b) contacting the double-stranded region of said hairpin oligonucleotide molecules with a mismatch binding protein; and
c) eliminating any molecules that bind to said mismatch binding protein, thereby producing a population of oligonucleotides that has reduced synthesis errors.

2. The method of claim 1, wherein said method comprises cleaving said loop region from said hairpin oligonucleotide molecules prior to step b).

3. The method of claim 1, wherein said eliminating is done by separating any molecules that bind to said mismatch binding protein from the remainder of the molecules by immobilizing said mismatch binding protein on a solid support.

4. The method of claim 1, wherein the mismatch binding protein is mutS or a variant thereof.

5. The method of claim 1, wherein the mismatch binding protein is T7 endonuclease 1 or a variant thereof.

6. The method of claim 1, wherein said mismatch binding protein is an endonuclease and said eliminating is done by cleaving said double-stranded region at the site of a mismatch.

7. The method of claim 6, wherein said endonuclease also cleaves said loop region.

8. The method of claim 1, further comprising, after the loop region has been cleaved, amplifying a sequence in said double stranded stem region using oligonucleotide primers that bind to the ends of said double stranded region of said hairpin oligonucleotides.

9. The method of claim 1, wherein said initial population of hairpin oligonucleotide molecules comprises multiple different hairpin oligonucleotides molecules that have the same loop region and the same or different double-stranded stem regions.

10. The method of claim 9, wherein the sequences of the double-stranded stem regions of said multiple different hairpin oligonucleotides are at least 80% identical to one another.

11. The method of claim 9, wherein said method further comprises assembling said reduced-error population of oligonucleotides into a plurality of different synthons, wherein said synthons are at least at least 80% identical to one another.

12. The method of claim 11, wherein said assembling is done by polymerase chain assembly (PCA) or ligase chain assembly (LCA).

13. The method of claim 1, wherein said hairpin oligonucleotide molecules comprise a site for a restriction enzyme in said double stranded region, proximal to said loop.

14. The method of claim 1, wherein loop region of said hairpin oligonucleotide molecules is at least four nucleotides in length.

15. The method of claim 1, wherein double-stranded region of said hairpin oligonucleotide molecules is at least 20 nucleotides in length.

16. A kit comprising:
a) a population of hairpin oligonucleotide molecules that each comprise a double-stranded stem region and a loop region; and
b) a mismatch binding protein.

17. The kit of claim 16, wherein the sequences of the double-stranded stem regions of said hairpin oligonucleotide molecules are at least 80% identical to one another.

18. The kit of claim 16, wherein said mismatch binding protein is T7 endonuclease I, mutS or a variant thereof.

19. The kit of claim 16, wherein said hairpin oligonucleotide molecules comprise a site for a restriction enzyme in said double stranded region, proximal to said loop and said kit further comprises said restriction enzyme.

20. A composition comprising:
a) a population of hairpin oligonucleotide molecules that each comprise a double-stranded stem region and a loop region; and
b) a mismatch binding protein;
wherein the mismatch binding protein is bound to any hairpin oligonucleotide molecules that have a synthesis error in the double-stranded stem region.
